(19) United States
(12) Patent Application Publication
(10) Pub. No.: US 2019/0142055 A1
(43) Pub. Date: May 16, 2019

(54) GRANULES

(71) Applicant: TUTTIFOODI B.V., Rossum (NL)

(72) Inventors: Gijs Robertus Johannes WEENINK, An Zevenaar (NL); Willem Hendrik VERMEER, An Zevenaar (NL); Andras Ferenc KUPECZ, An Zevenaar (NL)

(73) Assignee: TuttiFoodi B.V., Rossum (NL)

(21) Appl. No.: 16/089,046

(22) PCT Filed: Mar. 31, 2017
(86) PCT No.: PCT/NL2017/050204

§ 371 Date: Sep. 27, 2018

(30) Foreign Application Priority Data
Mar. 31, 2016 (NL) 2016523
Sep. 30, 2016 (NL) 2017557

(51) Int. Cl. A23P 10/22; A23L 27/10; A23L 27/40; A23L 23/10; A23F 3/32; A23C 13/12; A23L 27/12; A23K 40/10; A21D 10/00; A23L 33/26; A23P 30/30; A61K 8/02; A61K 8/73; A61K 9/16; A61Q 19/00

(52) U.S. Cl. CPC A23P 10/22; A23V 2002/00; A23L 27/40; A23L 23/10; A23F 3/32; A23C 13/125; A23L 27/13; A23K 40/10; A21D 10/005; A23L 33/26; A23P 30/30; A61K 8/0225; A61K 8/732; A61K 9/1694; A61K 9/1652; A61K 9/1688; A61Q 19/00; A23L 27/105

(57) ABSTRACT

The present invention relates to the preparation of granules and popped granules comprising functional substances, or active substances, and a carbohydrate carrier material, or matrix material, to the use of such granules, and the granules thus obtained. In the inventive preparation, a water-soluble dietary fiber is mixed with the functional substance in the presence of water or other aqueous solution, dried, and, after drying, converted in granules. The granules with improved properties thus obtained can be popped, providing popped granules with improved properties.

### BACKGROUND OF THE INVENTION

The present invention relates to the preparation of granules and popped granules comprising functional substances, or active substances, and a carrier material, or matrix material. Methods for the preparation of granulated products, in particular food products are well-known in the art.

Granulation is a technique of particle enlargement by agglomeration. During the granulation process, small fine or coarse particles are converted into large agglomerates called granules. Typically, preparation of granules involves wet granulation techniques and dry granulation techniques.

In wet granulation, typically, granules are formed by the addition of a granulation liquid (comprising the carrier/matrix) to the product to be granulated and under the influence of an impeller (in high-shear granulators), screws (in a twin screw granulator) or air (in a fluidized bed granulator). The agitation created by the granulator, in combination with the wetting of the components to be granulated results in aggregation of the materials, and thereby producing wet granules. Afterwards, the wet granules are dried, typically by spray drying. Thus, in the case of wet granulation techniques, the product to be granulated, that is either naturally wet, or artificially wetted, is in a pasty form at the time of granulation. The product to be granulated is moved around in a tank, for example by means of airflow, or on a spherical disk or with a blade rotor. In the granulation process, it continually receives a spray of carrier material, in liquid form to ensure agglomeration. In this method the carrier, once dissolved, should have a suitable viscosity to allow the solution containing the carrier to be pumped, in order to prevent clogging problems at the outlet of the injection nozzles, so as to promote the formation of fine droplets and to provide an even distribution thereof in the granulation tank, and at the same time a sufficiently high viscosity to allow the compound to play its role as carrier.

There are many wet granulation processes, see, for example, Powder Technology Handbook, Editor Lu Shouci. Chemical Industry Press, Beijing, 2004. For example, wet granulation processes include high-speed mixing granulation, extrusion-spheronization granulation, fluidized bed granulation, spray-drying granulation, compound granulation (agitation fluidized bed, rotation fluidized bed, agitation and rotation fluidized bed), and the like.

One particular type of wet granulation widely used in the art employs spray drying. Spray drying often is used as an encapsulation technique by the food and other industries. The substance to be encapsulated and a carrier are homogenized as a suspension in water. The resulting slurry is then fed into a spray drier, usually a tower heated to temperatures well over the boiling point of water. When the slurry enters the tower, it is atomized and forms micelles. The small size of the drops results in a relatively large surface area which dries quickly. As the water dries, the carrier forms a hardened shell around the load, creating the granules.

In dry granulation, typically, the dry granulation process is used to form granules without using a liquid solution because the product granulated may be sensitive to moisture and heat. In this process the primary powder particles are aggregated under high pressure. Dry granulator or a high-shear mixer-granulator can be used for the dry granulation.

Unfortunately, the methods available in the art suffer from various drawbacks, i.e. they are, to a varying level, complex, costly, operated at high process temperatures, inefficient, and require specialized, expensive equipment having a lot of cleaning time. Some methods in the art use potentially toxic carriers or matrix ingredients, whereas others require the use of free flowing or anti-caking agents or cross linking agents. Other drawbacks are low achievable flavor loads, poor water solubility of produced granulates and poor protection from oxidation by conventional granulates.

In view of the foregoing, it is clear there is a constant need for simple and improved methods for granulation of functional substances to provide a solution to the constraints of preparing granules with the methods in the art. In particular there is a need for granules, and methods for the preparations thereof, that improve the physical properties of such granules and allow, for example, for improved substance load, solubility, stability, e.g. resistance to oxidation (for example of the functional substance), flowability, flowability over time, without the need to use free flowing agents, use thermo sensitive substances, hygroscopicity properties (i.e. reduced), appearance, taste and odor, less demixing, the name a few.

In light of this, methods, products, compositions, and uses meeting one or more of such objectives would be highly desirable, but are not yet readily available. In particular there is a clear need in the art for reliable, efficient and reproducible methods of producing granules having improved characteristics. Accordingly, the technical problem underlying the present invention can be seen in the provision of such products, compositions, methods and uses for complying with any of the aforementioned needs. The technical problem is solved by the embodiments characterized in the claims and herein below.

### DESCRIPTION

### Drawings

Embodiments of the invention are further described hereinafter with reference to the accompanying drawings, in which:

FIG. 1 shows a close-up of a granule obtained with the method of the present invention. The granule in the photograph has not been popped (i.e. has been prepared after the mixture adopted the glassy state, without being “popped”).

FIG. 2 shows a close-up of the surface of a popped (i.e. expanded) granule obtained with the method of the present invention. As can be witnessed the surface of the popped granule appears continuous, without or with hardly any pores or cavities appearing therein, resulting in a robust granulate structure. The interior of the popped granulate typically has a cellular/honeycomb kind of structure, which enables the granulate to contain more volatile aroma components. This characteristic is generally observed for the popped granules prepared with the method of the present invention, in particular in case an indigestible dextrin is employed.

FIG. 3 shows a cross section of a popped granule according to the present invention. As can be witnessed, the interior of the granule display a cellular/honeycomb structure, or a meshwork structure with multiple pores or cavities dispersed within the meshwork, whereas the surface is a closed, without or with hardly any pores or cavities visible in the outmost layer, surface, of the granule.

FIG. 4 shows an example of a possible process flow for preparing granules with the method of the present invention. The skilled person understands various alternatives are possible, including additional steps and or excluding others. Where the process mentions the term “optional” the skilled person understands this is within the particular embodiment shown. Other steps not shown as optional may however be optional within the context of the current invention.

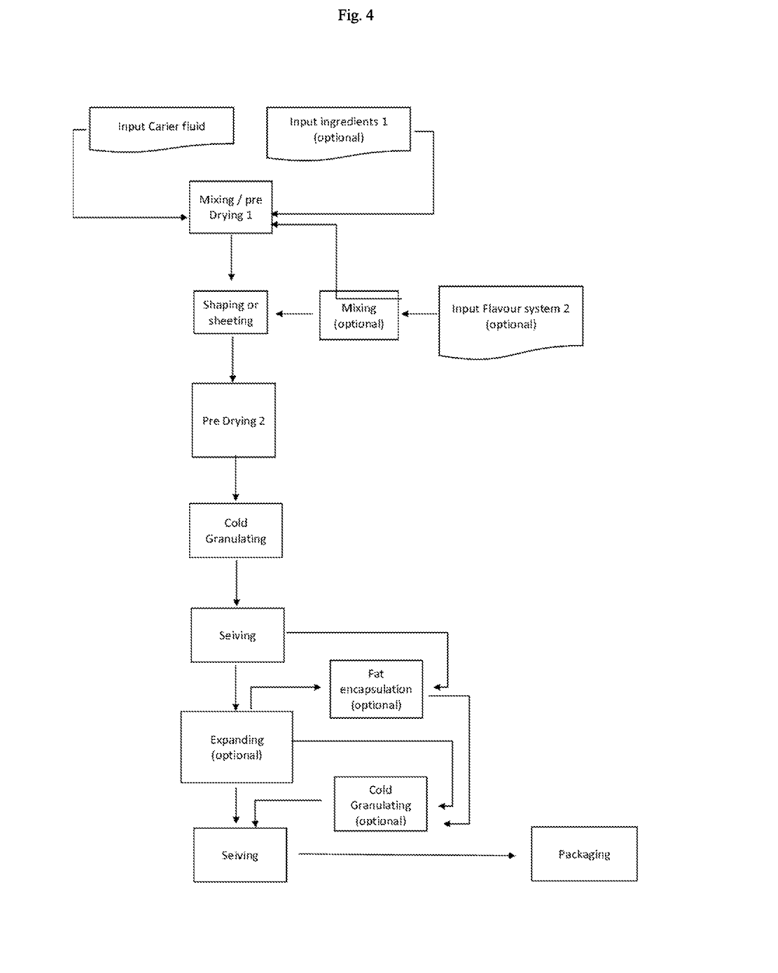

### DEFINITIONS

A portion of this disclosure contains material that is subject to copyright protection (such as, but not limited to, diagrams, device photographs, or any other aspects of this submission for which copyright protection is or may be available in any jurisdiction). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein.

“A,” “an,” and “the”: these singular form terms include plural referents unless the content clearly dictates otherwise. Thus, for example, reference to “a cell” includes a combination of two or more cells, and the like.

“About” and “approximately”: these terms, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

“And/or”: The term “and/or” refers to a situation wherein one or more of the stated cases may occur, alone or in combination with at least one of the stated cases, up to with all of the stated cases.

“Conventional techniques” or “methods known to the skilled person”: These terms refer to a situation wherein the methods of carrying out the conventional techniques used in methods of the invention will be evident to the skilled worker. The practice of conventional techniques in granulation, encapsulation and food techniques and related fields are well-known to those of skill in the art and are discussed, for example, in the following literature references: “Granulation” by Agba D. Salman, Michael Hounslow, Jonathan P. K. Seville; Elsevier (2006), and “Food Powders: Physical Properties, Processing, and Functionality” by Enrique Ortega-Rivas, Pablo Juliano, Hong Yan; Springer Science & Business Media (2006).

“Comprising”: this term is construed as being inclusive and open ended, and not exclusive. Specifically, the term and variations thereof mean the specified features, steps or components are included. These terms are not to be interpreted to exclude the presence of other features, steps or components.

“Glassy structure”: this term refers to the glass-like appearance of a material. The term “glass” or “glassy state” or “glassy matrix,” or “glassy structure” as used herein, refers to a liquid or liquid mixture that has lost its ability to flow, i.e. it is a liquid with a very high viscosity, wherein the viscosity ranges, for example, from 10¹⁰ to 10¹⁴ pascal-seconds (for example, as described by (or measured according to) Levine 2002, Amorphous Foods and Pharmaceutical Systems; The Royal Society of Chemistry). It can be viewed as a metastable amorphous system in which the molecules have vibrational motion and reduced rotational motion, but have very slow translational motion when compared to the liquid state. As a metastable system, it is stable for long periods of time when stored well below the glass transition temperature. The process used to obtain a glassy structure for the purposes of this invention is generally a solvent evaporation technique although other processes could produce a glassy matrix/structure. By way of example, glassy structure of several carbohydrates may also be obtained when a carbohydrate solidifies in a cooled carrier liquid. When the carbohydrate is in a glassy state, it exhibits an enhanced ability to protect encapsulated materials from vaporization and deterioration. The skilled person easily recognizes a glassy state within the context of the current invention as the material shows a glass-like appearance, is hard and brittle. A glassy state is adopted when the mixture losses plasticity and cannot be pumped like a liquid anymore.

“Nonflowable”: within the context of the current invention this term refers to a liquid or liquid mixture that has lost its ability to flow, but can still be pumped using mechanical pump. Within the context of the current invention a “nonflowable mixture” is still a liquid and not yet a “glassy structure”. Whereas the “glassy structure” is brittle and can be formed into granules, for example, by means of grinding or milling, the “nonflowable” mixture is a mixture that does not or hardly flow when put on a flat plate by 30 degrees Celsius, but is still plastic enough to be pumped. The nonflowable material can still be pumped as a liquid and still display plasticity.

“Popping or expanding”: The granules of the present invention may be converted to popped granules by popping the poppable granules of the present invention. “Popping” or “expanding” refers to a rapid, nearly explosive expansion of the granule, attended by a nearly instantaneous release of pressurized steam within the granule, often attended by an audible percussive sound. Upon heating of the granule, remaining water is heated past the boiling point and it forms a pressurized steam that is contained within the granule. The pressure continues to increase until the granule ruptures rapidly, causing a sudden drop in pressure in the granule and a corresponding rapid expansion of the steam, which expands the granule. As the granule rapidly cools, it forms an expanded or “popped” granule. Depending on the composition the granules may occupy 1.1 to 4 or more volume after popping. It may also be described as a shock solid-liquid-solid physical state transformation. By popping the granule, the specific volume per mass unit (number of cubic centimeters occupied by one gram of a substance) of the granule is increased, or said otherwise, the weight per volume will decrease, for example before popping the granules 100 gr/500 ml vs popped granule 80 gr/800 ml, and solubility of the granule is even further improved. Popping, within the context of the current invention, involves the rapid evaporation of remaining water from the granule, and preferably at the lowest possible temperature, preferably performed under reduced pressure conditions. Preparation of popcorn is an example of a method involving “popping”. Popping of the granules may for example, by achieved by using a conventional microwave, e.g. a 1000 W microwave, 20 seconds.

“Substance”: this term refers to a material with a defined chemical composition. A substance may be a liquid, fluid, or solid. The substance may consist of one (pure) compound or consist of a combination of compounds, i.e. a composition.

### DETAILED DESCRIPTION

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

As embodied and broadly described herein, the present invention is directed to a method for preparing granules comprising a functional substance and a water-soluble dietary fiber, the method comprising the steps of
a) preparing an aqueous mixture comprising said substance of interest and said water-soluble dietary fiber;
b) drying the mixture obtained in step a) until the mixture adopts a glassy structure; and
c) forming granules from the glassy structure obtained in step b).

The inventors of the present invention have surprisingly found that with the method of the present invention, granules may be obtained with desirable properties, such as increased solubility, hygroscopicity (e.g. reduced hygroscopicity; thereby no need for adding free flowing agents, even over time), flavor load (flavor loads of up to 90%, for example 20%, 30%, 40%, 50%, 60%, 70%, 75%, 80% may be achieved, in contrast to values of about 5-20% typically mentioned in the art), weight, taste and palatability, shelf life, resistance to degradation of the functional substance (e.g. by oxidation), and non-stickiness of the obtained granules using a simple, cost-effective and highly repeatable procedure.

In addition, the method of the present invention may be operated at low process temperatures, thereby preventing degradation or denaturation of thermo-sensitive compounds or ingredients. The temperatures that may be employed by the method of the present invention can be lower than used in traditional methods of preparing granules. For example, typical temperatures employed in spray-drying are well over 150 degrees Celsius, whereas the method of the present invention may be performed, for example at temperatures as low as 30-80 degrees Celsius, or even lower (e.g. as low as 10-80 degrees Celsius) For example when high (forced) airflow is used within the drying equipment, the glassy state may be formed at temperatures below 30 degrees Celsius.

Furthermore, the method for preparing granules does not require specialized, expensive equipment and that may have a lot of cleaning time. In the method, no toxic carriers or matrix ingredients need to be used and can be performed without the use of anti-caking agents or cross-linking agents.

The method of the invention can be easily adjusted or adopted depending on the desired requirements of the granule. In addition, the granules thus obtained can, surprisingly, be popped. The thus obtained popped granules display a high water-solubility and can show improved shelf-life. In addition, the popped granules display extreme good flowability, also in time (i.e. after long term storage) and without the need to use free flowing agents such as siliciumdioxide and magnesium stearate.

The method of the invention allows providing uniform granules in one process, without the need of additional granulation steps, and while producing less waste material. The method also allows the use of (partially) insoluble materials, i.e. granules can be prepared from aqueous mixtures (as described below) comprising non-solubilized materials (Depending on the size of the granule, the non-solubilized or solid materials may, for example have a size no more than 50, 100, 250 or 500 micrometer). Examples are vegetables, fruit, spices, herbs and other finely pureed or chopped materials.

The method of the invention is quick and reproducible, making it very suitable for, for example, preparing granules comprising flavors and perfumes, in particular volatile flavors and perfumes. In studies, it has been found that, for example, flavorings and/or perfumes can be sufficiently granulated in a short time span, and that, secondly, a product is obtained that is improved with respect to the distribution of particle sizes, the geometry, the retention and loading. A high loading here means a high total amount of encapsulated substance of interest, e.g. flavoring, based on the granule mass. The higher the retention of the individual components, in particular volatile substance, the lower are the losses of these components.

The method of the present inventions can be described as a method that encapsulates substances of interest in a matrix formed by a water-soluble dietary fiber. By combining the water-soluble dietary fiber with the substance of interest, the substance of interest will become intimately mixed with the matrix material or carrier material, i.e. the water-soluble fiber. In the granules of the present invention both the dietary fiber and the substance of interest are typically essentially homogenously dispersed throughout the granule.

In a first step of the method, an aqueous mixture is prepared comprising the functional substance and the water-soluble dietary fiber (herein also referred to as “water-soluble fiber” or “fiber”).

The aqueous mixture may be provided in any volume. In practice mixture, and preferably, amounts of the aqueous mixture of at least 1 liter, at least 10 liters, at least 100 liters, at least 1000 liters or more are prepared.

For the aqueous mixture, the type of water-containing liquid that may be used is not in particular limited. Examples of suitable liquids include demineralized water, or plain tap water.

In the present invention, the term “substance of interest” refers to the substance that is to be incorporated in the granule together with the water-soluble dietary fiber, in other words, a functional substance. The term functional is used to denote the substance is selected as such to be included in the granule, and preferably is edible or can be digested and/or metabolized e.g. by a human or animal body. The substance of interest (also referred herein as the “substance”) refers to a defined chemical composition. The substance of interest may be a liquid, fluid, or solid, preferably the substance used in the present invention is a solid. The substance of interest may consist of one (pure) compound or by consist of a combination of compounds, i.e. a composition.

The method of the present invention is preferably for the production of granules as an ingredient in a food, a feed, a pharmaceutical or a cosmetic. Therefore, preferably, the substance of interest is a food substance, a feed substance, a pharmaceutical substance or a cosmetic substance, i.e. a compound or composition that is or forms part of a food, feed, pharmaceutical or cosmetic.

Examples of product and compositions for which the granules prepared with the method of the present invention are useful include, but are not limited to, vegetables and fruit, or combinations of vegetables and fruit, preserving the aroma, nutrients and color in a granulate format with a long ambient shelf life; instant granulates for cold beverages such as inter alia instant fruit juices, ice tea, vitamin waters, energy beverages or smoothies, but also soft drinks; instant granulates for hot drinks such as inter alia instant tea or coffee variants; flavoring systems such as seasoning varieties, as may be used on e.g. paprika chips or on salads; instant bouillons, soups and sauces; sweeteners, such as inter alia table sugars or sugar substitutes; creamers, such as inter alia coffee milk powders; dried plant extracts or herbs and spices; baby foods; vitamin, mineral and nutrient compositions; vegetable and fruit granulates kneaded and compressed into vegetable and fruit hard and soft candies; personal care materials, such as inter alia hand soap based on granules, bath pearls; and pharmaceutical granules, tablets or powders. Examples of finished goods instant application areas include but are not limited to baby foods, vegetable and/or fruit drinks, soups, sauces, bouillons, beverage base mixes, coffee, tea, coffee creamers, table top sweeteners, condiment toppings.

Therefore, according to one preference, the substance of interest is a substance that is or forms part of such products for, or in, which the granules obtained with the method of the present invention may be used.

Preferably, the substance to be used in the method of the present invention is an edible compound or composition. Example include, but are not limited to coffee concentrates, cocoa, dairy products, extracts and distillates, flavoring agents (hydrophilic and lipophilic), fats or oils, food, food extracts, fragrant, herbs, meat, meat extracts, milk powders or condensates, minerals, phytonutrients, plant extracts, proteins, spices, tea, tea extracts, vitamins, and combinations thereof. In addition, the substance of interest can be a biologically active substance or organism, such as yeast, bacteria, starter cultures, enzyme, therapeutic protein, such as a vaccine or antibody, and other biological substances, including combinations thereof.

The substance to be encapsulated or included in the granules is mixed with a water-soluble dietary fiber.

In the present invention, the term “water-soluble dietary fiber” refers to any type of water-soluble indigestible (for humans) saccharide, i.e. saccharides that are poly- or oligosaccharides, which “attracts” water. Water solubility can be seen as meaning that at least 10 or 20 grams can be dissolved in 100 ml water at 20 degrees Celsius. Water-soluble fibers may be dissolved in water to make a clear solution. Water-soluble fibers are not or only to a minor extent digestible by humans, but are, typically, fermented by bacteria present in the gastro-intestinal tract. Water-soluble fibers may induce the growth or activity of microorganisms in the gastro-intestinal tract, and are therefore also sometimes referred to as (water-soluble) prebiotic fibers.

The water-soluble dietary fibers may roughly be divided into high viscous ones and low viscous ones. Examples of high viscous water-soluble fibers include pectin, powdered konjak (mannan), alginic acid salts, propylene glycol ester of alginic acid, guar gum, and agar. These types are used in industry as thickeners. In a preferred embodiment, these types are not preferred, for example since the types typically do not have a neutral odor, color, or flavor profile and are not transparent, and may be more difficult to form into a glassy structure.

Examples of low viscous water-soluble fibers include digestion-resistant dextrins (also referred to as indigestible or resistant dextrins, digestion-resistant maltodextrin (also referred to as branched maltodextrin, indigestible or resistant maltodextrins)), polydextrose, inuline, or combinations thereof. Typically, the low viscous water-soluble fibers can be dissolved in water in amount of no less than 10 g, preferably 20 g in 100 ml at 20° C. Typically, the viscosity of a 5% (w/w) mass aqueous solution thereof is less than 20 mPas at 20° C. (measured for example using a Brookfield RV with appropriate spindle and RPM (e.g. spindle 4, 20 RPM)). Concrete commercially available examples of those exemplified above include, but are not limited to “Fibersol-2” manufactured by ADM/Matsutani Chemical Industry Co., Ltd.; “Litesse” manufactured by Danisco Cultor; and “Nutriose” manufactured by Roquette.

Preferably, the water-soluble fiber is a low viscous water-soluble fiber, preferably is a low viscous water-soluble fiber selected from digestion-resistant dextrins, such as Nutriose and Fibersol, (also referred to indigestible or resistant dextrins, digestion-resistant maltodextrin (also referred to as branched maltodextrin, indigestible or resistant maltodextrins), polydextrose, inuline, or combinations thereof. Digestion resistant can be seen as denoting that the dextrin or maltodextrin contains non-digestible linkages, such as (1,2) and (1,3)-glucosidic linkages. These non-digestible linkages are not hydrolyzed by human digestive enzymes, or at least not to a substantial degree. The terms digestion-resistant dextrin and digestion-resistant maltodextrin are very well-known to the skilled person.

The term water-soluble dietary fiber may also be considered to include various oligosaccharides such as fructooligosaccharides, galactooligosaccharides and xylooligosaccharides. However, these types of (e.g. having a degree of polymerization of 2-9 or 2-6) are less suitable for use in the method of the invention and are not preferred. As the skilled person knows, the degree of polymerization, or DP, is defined as the number of monomeric units in the polymer or oligomer.

Preferably, the water-soluble dietary fiber is a water-soluble dietary fiber (preferably a polymeric water-soluble dietary fiber, even more preferably a low viscous polymeric water-soluble dietary fiber) with a degree-op-polymerization (DP) of more than 8, preferably more than 10. For example, the degree of polymerization is between 8-80, preferably between 10-70, even more preferably between 12-60.

The water-soluble dietary fibers suitable for use in the method of the present invention form a glassy structure upon drying of an aqueous mixture comprising/containing said dietary fiber; as exemplified herein.

Although dietary fibers suitable for the present invention are water-soluble fibers, minor amounts of water-insoluble fibers can be included (examples of which include cellulose, some hemi-celluloses, fibers that may be obtained from wheat bran, apples, and chitin).

The aqueous mixture comprising the substance of interest and water-soluble dietary fiber may be prepared according to any method known to the skilled person.

The preparing of the aqueous mixture may be performed at room temperature, or the aqueous medium may be heated or cooled before or during adding the substance of interest and the water-soluble dietary fiber. Preferably, the mixture is prepared at a temperature between 10-80 degrees Celsius, preferably between 20-60 degrees Celsius.

For example, the substance of interest may first be added to the aqueous medium, followed by addition of the water-soluble dietary fiber. The substance and fiber may also first be mixed and subsequently be added to the aqueous medium.

Alternatively, the water-soluble dietary fiber is first mixed with the aqueous media, for example at an elevated temperature, after which the substance of interest is added to the water-soluble fiber containing aqueous mixture, for example after cooling down of the mixture. This also includes the situation wherein first the water-soluble dietary fiber is mixed with the aqueous media, and before the substance of interest is added, part of the water-content of the aqueous mixture comprising the water-soluble dietary fiber is removed, for example by evaporation, followed by the addition of the substance of interest before the mixture adopts a glassy structure. This is for example desirable in case the soluble dietary fiber is added to the aqueous mixture at a temperature that is not desirable (for example due to thermosensitive) for the functional substance.

If so desired, substances of interest and/or soluble dietary fibers may also be provided to the mixture at various time point and/or temperatures of preparing the aqueous mixture.

In other words, step a) of the method of the present invention provides a mixture of the substance of interest and the water-soluble dietary fiber before the mixture adopts a glassy structure.

The relative amount of substance of interest and/or soluble dietary fiber is not in particular limited in the method of the present invention. Preferably the amount of soluble dietary fiber used in preparing the mixture is such that all soluble dietary fiber dissolves (disperses) in the aqueous media.

For the present invention, it is not required the functional substance is dissolved, in part or completely, in the aqueous media. For example, the functional substance may consist of or comprise water-insoluble materials, or materials that are not dissolved within the time for preparing the aqueous mixture. Obviously, under such circumstances measures are taken to ensure a more or less homogenous distribution of the materials disperses throughout the mixture. Such insoluble material may typically be up to 500 micrometers in seize, and generally should not have a particle size bigger than half of the seize of the granule, in particular of the glassy state granule, i.e. before popping.

Typically, the aqueous mixture obtained in step a) may have a relative amount of water (expressed as weight percentage of the total weight of the aqueous mixture) over a broad range, for example from 5-99.9% (w/w), preferable from 10-60% (w/w), more preferably from 10-50% (w/w), or even from 10-40% (w/w). Preferably the aqueous mixture obtained in step a) has a water content of at least 10% (w/w).

The aqueous mixture thus obtained will, in a next step of the method, be dried, as a mixture, until the mixture adopts a glassy structure. In this step water is allowed to part from the mixture. This may be achieved by any possible means, for example using drying is by convection drying, conduction drying, radiation drying, or combinations thereof. For example, drying may be with a microwave oven, for example under reduced pressure (as compared to atmospheric pressure). On particular method of drying may involve pulsed drying using a microwave oven, for example, under reduced pressure. In such method, the mixture is subject to drying with the microwave for a short period of time, for example the microwave is turned on for a period of 5-10 seconds, followed by a period wherein the microwave is off, and followed by a next short period wherein the microwave oven is turned on, and repeated until the mixture adopt to glassy structure. This prevent the temperature in the mixture to raise to undesirable height and improves overall quality of the products obtained.

Depending on the relative amount of water and the viscosity of the mixture, part of the water may initially be removed, preferably while still stirring the mixture (for example, in order to keep the substance of interest and/or the water-soluble dietary fiber homogenously mixed throughout the mixture). Removal may be applying heat, for example, under reduced pressure, as described herein. Once the mixture becomes viscous it may become less necessary to stir the mixture. This is for example, typically the case when the relative amount of water (by weight) in the mixture is reduced, for example to 5-20% (w/w), or 8-15% (w/w). For example, at such relative amounts of water, a mixture is obtained that is highly viscous and stops flowing e.g. when put on a plate, e.g. at a temperature below 30 degrees Celsius, i.e. the mixture becomes a non-flowing mixture (see also below), although it has not yet adopted a glassy structure as defined herein.

The skilled person knows that this value may depend on the type(s) of soluble dietary fiber, the substance of interest, and the (relative) amounts thereof, used in preparing the initial the aqueous mixture.

In order to allow the aqueous mixture to adopt a glassy structure, the aqueous mixture, should at the last phase of the drying in step b) not be stirred or mixed.

Removal of water may be achieved by any method known to the skilled person, for example by heating the mixture, for example under reduced pressure (e.g. anywhere between 10⁵-10⁻⁵ Pa). The skilled person understands what method and equipment can suitably be used, including vacuum dryers, microwaves and/or belt dryers.

One of the major advantages of the method of the present invention in comparison to those in the prior art is that the method of the present invention allows the use of moderate to low temperatures for drying the aqueous mixture. Although drying temperature is not in particular limited, it was found that granules obtained after drying of the aqueous mixture at a temperature of at most 90 degrees Celsius, at most 80 degrees Celsius, at most 70 degrees Celsius, at most 60 degrees Celsius, at most 50 degrees Celsius, at most 40 degrees Celsius, at most 30 degrees Celsius displayed the best properties. Thus, according to one preference, drying of the mixture is at a temperature of between 5-90 degrees Celsius, preferably 10-80 degrees Celsius. Thus, drying temperature in a preferred embodiment may be at a temperature between 5, 10, 15, 20, 25, 30 degrees Celsius as the low-end temperature and 40, 50, 60, 70, 80, 90 degrees Celsius as the high-end temperature (in any possible combination of the low end and high end temperature).

Water may be removed from the aqueous mixture of step a), e.g. by evaporation, while the aqueous mixture is being held in a container and until the mixture adopts a glassy structure. When the aqueous mixture has obtained, or is provided with, a viscosity that allows casting of the aqueous mixture, or the forming of a shape, the mixture may also be dried in the form of such shape, for example a sheet. For example, the aqueous mixture may be casted in the form of a layer, after which the layer is dried until it adopts the glassy structure.

Once the glassy structure is obtained, i.e. when the mixture has become hard and brittle, drying may be stopped, or, if so desired may be continued for an additional period of time.

Typically, the glassy structure obtained in step b) will, preferably, have a relative amount of water (expressed as weight percentage of the total weight of the glassy structure) over a broad range, for example from between 0.01-15% (w/w), between 0.05-10% (w/w), between 0.1-8% (w/w) or between 0.1-5% (w/w). The skilled person knows that this value may depend on the type(s) of soluble dietary fiber, the functional substance, and the (relative) amounts thereof, used in preparing the initial the aqueous mixture.

Drying time will depend on the temperatures and pressure (vacuum) used for drying, as well as the initial water content and layer thickness. Drying of the mixture may be as short as only a few minutes or may be for several hours (e.g. anywhere between 2 minutes-8 hours-even 14 hours or more).

It is for the present invention in particular relevant to note that drying within the context of the present invention preferably involves the drying of the aqueous mixture as obtained under step a) as a whole. Drying according to the method of the present invention is preferably different to methods in the art. The methods in the art typically rely on techniques that first require the formation of small drops or a spray. In such spray drying, chilling or cooling techniques the mixture as a whole is not dried, but the mixture is first sprayed, thereby obtaining a spray, which is then dried. For example, the current invention, preferably, employs plate drying of the mixture, and preferably at temperatures below 90 or 80 degrees Celsius, followed by separate granulation, in contrast to the art, employing drop or spray formation, followed by drying as a mean to obtain granules.

In other words, the method of the present invention preferably does not require or depend on the formation of small drops or a spray of the obtained mixture. The method of the present invention allows the direct drying of the aqueous mixture obtained in step a). Clearly the skilled person understands that “drying the aqueous mixture” also includes the direct drying of a part of the aqueous mixture obtained in step a), for example at least 1% (w/w), 10% (w/w), 20% (w/w) or more of the aqueous mixture obtained in step a).

After the aqueous mixture of step a) has been dried in step b) until the mixture adopts a glassy structure, the thus obtained glassy structure may be formed into granules. It was found that with the method of the present invention, the glassy structure can easily be formed into granules using commonly available techniques, such as, but not limited to breaking and milling techniques.

Preparing the granules from the glassy structure may be performed at any temperature, but is preferably performed at a temperature between 0-40 degrees Celsius.

Depending on the desired specification of the granules, the granules may be sieved, for example using one or more sieves, until a desired particle size of particle size distribution is obtained. With the method of the present invention it is possible to provide granules with a wide range of maximum diameters (size). Although not limited thereto, preferably the size of the granules is in the range of 50 micrometers-5000 micrometers, preferably from 100-2000 micrometer. The granule size is as determined by passing through a mesh sieve. The particle size refers to the diameter of a globe or the equivalent circle diameter of a particle having a non-global shape.

After the granules are formed, they may be further treated, if so desired. For example, the granules may be coated with a second layer. For example, the granules may be coated with a coating that prevents instant solubility of the granules. This may be advantageous is case, for example, the granules are to be taken up in water-based emulsions. By applying, for example, a fat coating around the granule, instant solubility is prevented. This allows (heat stable) use of the granules of the invention is, for example, hot oven or (deep) frying applications.

However, according to a highly preferred embodiment, the granules obtained with the method of the present invention are treated so as to rapidly expand the granule. This popping of the granule may be performed using any method known to the skilled person.

Thus, in such preferred method of the present invention, the method further comprises a step d) of popping (or expanding) at least part of the granules obtained in step c) to obtain popped (or expanded) granules. As will be described herein the granules thus obtained have unique and unexpected properties, not limited to an even better solubility in comparison to the non-popped granules and/or a reduced hygroscopicity in comparison to non-popped granules and/or are free flowing, even over time, and in the absence of free-flowing agents.

Preferably, the granule that is popped is a granule comprising as the or as one of the soluble dietary fiber digestion-resistant dextrin or digestion-resistant maltodextrin, such as Nutriose, e.g. Nutriose FM06/FB06 and/or Fibersol-2.

By rapidly evaporating fluid, the particle will expand or pop. Depending on the degree of expansion, the solubility in hot or cold water of the granule can be regulated. The solubility may be enhanced by increasing the degree of expansion of the granule. The degree of expansion determines the dissolution rate.

Preferably popping is performed by use of a fluid bed dryer, a plate dryer, a hot plate, a belt dryer, a spray dryer, a microwave, or by applying radiation, including drying by non-continues energy input, for example, using microwave technology and/or wherein popping is performed under reduced pressure, for example at a reduced pressure anywhere between 10⁵-10⁻⁵ Pa.

Preferably popping/expanding of the granules is performed at reduced pressure. Preferably popping is performed in a short period of time. Preferably popping is performed between 1 second and 5, 4, 3, 2, 1 minutes, 50 seconds, 40 seconds, 30 seconds, 20 seconds. The skilled person understands how to establish the conditions to expand, i.e. pop, the granules obtained with the method of the present invention.

To keep the substance of interest, for example a flavor profile from odor and flavor substances in the granule, the skilled person understands that using an as low as possible temperature may be desirable, for example at temperature between 0-80, or 0-50 degrees Celsius, and preferably in combination with a reduced pressure, e.g. drying under high or low vacuum, e.g. anywhere between 10⁵-10⁻⁵ Pa. Alternatively, higher temperatures may be used.

Typically, the popped granule obtained in step d) will, preferably, have a relative amount of water (expressed as weight percentage of the total weight of the glassy structure) over a broad range, for example, and with increasing preference, between 0.001-12% (w/w), between 0.005-11% (w/w), between, or 0.01-10% (w/w).

The skilled person knows that this value may depend on the type(s) of soluble dietary fiber, the substance of interest, and the (relative) amounts thereof, used in preparing the initial the aqueous mixture.

Preferably, the water-soluble dietary fiber is digestion-resistant dextrin, polydextrose, digestion-resistant maltodextrin, inulin or combinations thereof. Even more preferably the water-soluble dietary fiber is digestion-resistant dextrin or digestion-resistant maltodextrin, even more preferably the water-soluble dietary fiber is Nutriose, even more preferably Nutriose type FM06/FB06 or FM10 (derived from maize) or FB06/FB06 or FB10 (derived from wheat).

Polydextrose is a water-soluble, low calorie non-cariogenic bulking agent. Polydextrose (for example available under the trade name Litesse™ from Danisco) is a soluble, random polymer of dextrose containing minor (less than about 10 wt. %) amounts of sorbitol (at least 2 wt. %) and citric acid. Typical polydextrose polymers contain around 10 to 50 saccharide units, preferably 20 to 40 saccharide units. Polydextrose is, for example, available from Tate&Lyle as Sta-Lite L90, Sta-Lite R90, from Danisco as Litesse Ultra™ IP powder.

Inulin is a group of oligosaccharides occurring naturally in many plants and belongs to a class of carbohydrates called fructans. Inulin is a prebiotic fermentable fiber and is metabolized by the gut. Inulin is composed of linear chains of fructose units linked by β (2→1) bonds and is often terminated by a glucose unit. Inulin sources contain polymers in a distribution of chain lengths, which are described by their DP (degree of polymerization). Typically, short chain linear inulin has DP<20 and long chain linear inulin has DP>20. A typical long chain linear inulin source, such as Beneo HP inulin supplied by Orafti, has an average DP>23. A typical short chain linear inulin source, such as DeSugar Inulin supplied by Cargill, has an average DP=10, creating an inulin material with short polymer chains. Sensus inulin is sold under the name Frutafit CLR.

Among the water-soluble dietary fibers suitable for use in the method of the present invention, the digestion-resistant dextrin is most effective and preferred. Digestion-resistant dextrins are partially hydrolyzed starches (glucose polymers) for example produced by heating starch in the presence of small amounts of food-grade acid.

Dextrinization results in a drastically reduced molecular weight and the introduction of new glucoside linkages. Digestion-resistant dextrins contain nondigestible (1,2) and (1,3)-glucosidic linkages. These non-digestible linkages are not hydrolyzed by human digestive enzymes. As a result, a portion of the dextrin is not digested in the upper part of the gastro-intestinal tract and is not directly available as such for energy utilization. However, a portion of the non-digested material is hydrolyzed by bacterial flora in the colon and the resulting free fatty acids are utilized for energy. The occurrence of non-digestible linkages is a well-known characteristic of digestion-resistant dextrins as well as polydextrose. In view thereof, a digestion-resistant dextrin is particularly characterized by being a partially hydrolyzed starch (glucose polymer); having nondigestible (1,2) and (1,3)-glucosidic linkages; and/or having an average molecular weight of 3500-6500 dalton.

In addition to digestion-resistant dextrins, also digestion-resistant maltodextrins are working quite well. Resistant maltodextrins are made from starch by pyrolysis and subsequent enzymatic treatment to convert a portion of the normal α-1,4 glucose linkages to random 1,2-, 1,3-α or β linkages. In view thereof, a digestion-resistant maltodextrin is particularly characterized by having 1,2-, 1,3-α and/or β linkages; and/or having an average molecular weight of 3500-6500 dalton.

Preferably the water soluble fiber is a digestion-resistant dextrin, such as Nutriose, preferably Nutriose FM 06 and/or FM10, or second-best a digestion-resistant maltodextrin, such as Fibersol-2

As can be witnessed from the example, use of digestion-resistant dextrins, e.g. Nutriose FM06/FB06, or digestion-resistant maltodextrin, such as Fibersol-2 provides for granules and expanded or popped granules that meet many of the desired requirements, including, but not limited to fast solubility of the digestion-resistant dextrin or digestion-resistant maltodextrin, and ease of use in preparing the granules, which are free flowing, non-sticking, neutral in taste, and fast soluble. The granules obtained with digestion-resistant dextrins, or a digestion-resistant maltodextrins, can easily be popped (expanded), in a fast way, yielding non-sticking popped granules which are free flowing and fast soluble.

The skilled person understands that also mixtures of different water-soluble dietary fibers may be used in the method of the present invention. For example, 1, 2, 3, 4 or more different types of water-soluble fibers may be used. For example, polydextrose may be mixed with an digestion-resistant dextrin or a digestion-resistant maltodextrin, in any ratio, for example in a ratio of 1:20.

Preferably, the water-soluble dietary fiber is a water-soluble dietary fiber (preferably a polymeric water-soluble dietary fiber, even more preferably a low viscous polymeric water-soluble dietary fiber) with a degree-op-polymerization (DP) of more than 8, preferably more than 10. For example, the degree of polymerization is between 8-80, preferably between 10-70, even more preferably between 12-60.

In case the water-soluble dietary fiber is an digestion-resistant dextrin, such as Nutriose, e.g. Nutriose FM06 or FM10 or FB06 or FB10, or a digestion-resistant maltodextrin, such as Fibersol-2, preferably has an average degree-of-polymerization of 4-30, for example 8-27 or 9-25, preferably the average degree-of-polymerization is 4-10 or 12-25.

Preferably the digestion-resistant dextrin, preferably Nutriose, or a digestion-resistant maltodextrin, such as Fibersol-2, has an average molecular weight of 3500-6500 dalton, for example 3500-4500 dalton or 4000-6000 dalton.

Preferably, the water-soluble dietary fiber is a digestion-resistant dextrin with an average degree-of-polymerization of 4-30, for example 8-27 or 9-25, preferably the degree-of-polymerization is 4-10 or 12-25 and/or an average molecular weight of 3500-6500 dalton, for example 3500-4500 dalton or 4000-6000 dalton.

The skilled person knows how to determine the degree-op-polymerization and/or the average molecular weight of the water-soluble dietary fiber, for example of the digestion resistant dextrin, using methods available in the art.

As already discussed above, the relative amount of water used in preparing the aqueous mixture in step a) may vary over a broad range. The skilled person will however understand that the aqueous mixture should eventually be dried (i.e. water should be removed) in order to allow the mixture to adopt the glassy structure required for preparing the granules. It was found that with the method of the present invention there is no need to initially use relative high amount of water in the aqueous mixture.

Therefore, preferably the mixture prepared in step a) is prepared as a non-flowable mixture, i.e. a mixture that, when poured or put on a plate, for example at 30 degrees Celsius, does not flow. Alternatively, and as discusses above step a) comprises a step of removing water from the mixture prepared in step a) to provide for such non-flowable mixture. Such non-flowable mixture can still be pumped, e.g. using electric pumps but does not flow or spread out on its own motion, for example when a layer of 1-3 cm is placed on a horizontal orientated plate. The skilled person understands that water may be removed as discussed above.

As mentioned above, the mixture prepared in step may be formed into a shape, preferably a sheet prior to the forming of the glassy structure in step b). Indeed, depending on different end-applications and functional requirement the mixture can be shaped in a variety of unique formats, i.e.: round, rectangular, spiral- or needle-shaped.

More in particular, a highly viscous mixture may be formed into a shape by casting and/or injection molding. Preferably the casting or molding is on a non-sticky surface, such as a silicone layer, Teflon or a smooth surface. Preferably the mixture has a viscosity such that the shape essentially preserves its shape after casting and/or molding without the need for supporting boundaries or edges. For example, the mixture used to prepare the shape is a non-flowable mixture.

Preferably, in case sheet is formed, the sheet has a thickness that allows easy and quick drying, preferably at moderate temperatures, in the next step of the method. Preferably, the sheet is provided with a sheet thickness of, with increasing preference, between 0.01-20 cm, between 0.05-10 cm, between 0.1-4 cm, or between 0.1-2 cm. It was found that such sheets allow for easy, quick drying and provides excellent granules, in particular when a digestion-resistant dextrin, such a Nutriose, or a digestion-resistant maltodextrin, such as Fibersol-2 is used.

As discussed above, the functional substance to be included in the method may be any type of substance. Preferably, the functional substance is a food substance, a feed substance, a pharmaceutical substance or a cosmetic substance.

Preferably, in step a) the functional substance and the water-soluble dietary fiber are mixed in a weight ratio 1:20-20:1, or even 1:100-100-1. For example, the weight ratio may be 1:100, 1:50, 1:20, 1:15, 1:10, 1:5, 1:1, 5:1, 10:1, 15:1, 20:1, 50:1, 100:1. The skilled person understands that suitable weigh ratios will depend on the substance and fiber used in preparing the mixture as well as on the desired properties of the end product. It was however found that the method of the invention allowed weight ratio's to be prepared over a broad range, while preserving important properties of the granules, such a solubility, reduced hygroscopicity, and others and mentioned herein (or while only showing minimal reduction in such properties).

As already described above, one of the major achievements of the current invention is that it allows preparing granules while employing temperatures that are moderate, allowing to preserve the properties and characteristics of the functional substances encapsulated by the method. Therefore, preferably the method involves drying at such moderate temperature. Preferably, in step b) drying is at a temperature of, with increasing preference, at most 90 degrees Celsius, at most 80 degrees Celsius, at most 70 degrees Celsius, at most 60 degrees Celsius, at most 50 degrees Celsius, or at most 40 degrees Celsius. Preferably drying at the temperatures indicated is in combination with drying under reduced pressure, as already discussed herein.

Preferably in step b) drying is under conditions with a relative humidity of, with increasing preference, at most 50%, at most 40%, at most 30%, or at most 20%.

Preferably the glassy structure obtained in step b) may have a relative amount of water (expressed as weight percentage of the total weight of the glassy structure) over a broad range, for example from between 0.01-15% (w/w), between 0.05-10% (w/w), between 0.1-8% (w/w) or between 0.1-5% (w/w).

The skilled person knows that this value may depend on the type(s) of soluble dietary fiber, the functional substance, and the (relative) amounts thereof, used in preparing the initial the aqueous mixture, as well as the duration of drying. For example, after the glassy structure is obtained, drying may continue, thereby further reducing the relative amount of water in the glassy structure.

After the glassy structure is obtained, granules may be prepared, for example by (correct) breaking or milling the glassy structure obtained in step b). After breaking and milling the glassy granules or pieces may be sieved out by means of different sieves and mesh sizes, for example, until the desired particle size distribution is obtained. Depending on the desired product characteristics, these granules may be used as is and/or be popped (expanded), as described above.

The granules or popped granules may, for example, be used directly in or as seasonings. It is also possible to treat the granules/popped granules by using knives in order to provide a fine powder.

Different granules, which may be the result of the different steps of the method provided herein, may be mixed to prepare mixtures with unique characteristics, properties and flavor profiles. The granules may be packed separately or be included in various products, such as these described herein.

As mentioned above, preferably, the popped granules obtained in step d) have a water content of, with increasing preference, between 0.001-12% (w/w), between 0.005-11% (w/w), between, or 0.01-10% (w/w).

Also provided is a granule or popped granule obtainable or obtained with the method of the present invention.

In particular, there is provided a granule or popped granule, comprising, by weight,
1-99%, preferably 40-99% substance of interest, even more preferable 75-99% or 40-75%;
1-99%, preferably 1-60%, even more preferably 1-25% or 25-60% or up to 10, 11, 12, 13, 14, 15, 16, 17, 18, 19, 20, 21, 22, 23, 24, 25% water-soluble dietary fiber; and
0-15% water, and wherein substance of interest, water-soluble dietary fiber and water add up to no more than 100%, preferably 100%. Preferably, the water-soluble dietary fiber is a digestion-resistant dextrin, such as Nutriose or a digestion-resistant maltodextrin, such as Fibersol-2

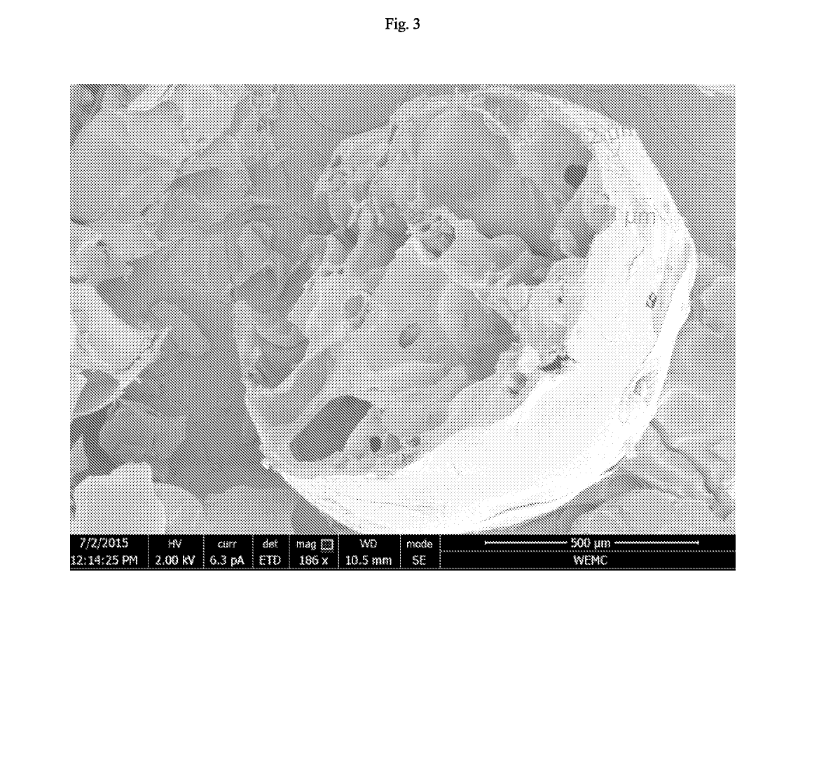

Also provided is for a popped granule, comprising, by weight,
1-99%, preferably 40-99% substance of interest, even more preferable 75-99% or 40-75%;
1-99%, preferably 1-60%, even more preferably 1-25% or 25-60% or up to 10, 11, 12, 13, 14, 15, 16, 17, 18, 19, 20, 21, 22, 23, 24, 25% water-soluble dietary fiber; and
0-15% water, wherein substance of interest, water-soluble dietary fiber and water add up to no more than 100%, preferably 100%, and wherein the popped granule is a granule characterized by an outer surface that is substantially closed and an interior with a cellular structure or meshwork structure with multiple pores or cavities dispersed within the meshwork, for example a honeycomb structure, preferably wherein the granule has a size of between 50 micrometer and 5000 micrometer, more preferably between 100 micrometer and 2000 micrometer, preferably wherein the pores or cavities in the interior are present in a quantity of 15 to 90%, for example 15-75% by volume. Preferably the water-soluble dietary fiber is a digestion-resistant dextrin, such as Nutriose or a digestion-resistant maltodextrin, such a Fibersol-2. The outer surface layer may have, for example, a thickness of about 3-40 micrometers (see FIG. 3).

For both the granules or the popped granules, the weight percentage of functional substance may vary between 1-99%, and may, for example be 1-5%, 10-50%, 20-80%, 40-75%, or 30-95%, or 40-95%, 50-95%, 90-99%.

For both the granules or the popped granules, the weight percentage of the soluble dietary fiber may be between 1-99%, and may for example be 1-5%, 10-50%, 20-80%, 40-75%, or 30-95%, or 40-95%, 50-95%, 90-99%.

Preferably water content is less than 14, 13, 12, 11, 10, 9, 8, 7, 5, 4, 3, 2, 1% (w/w).

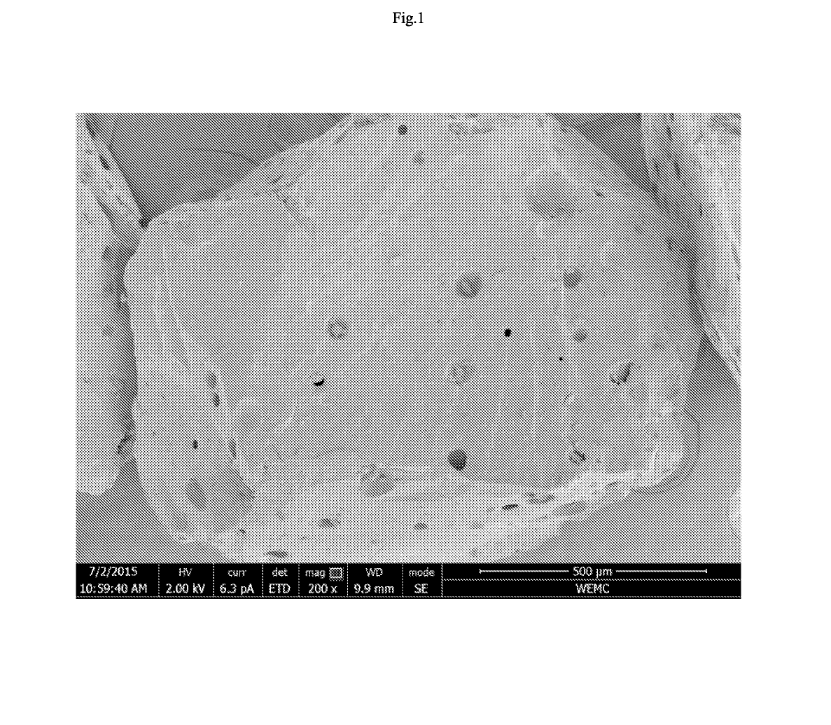

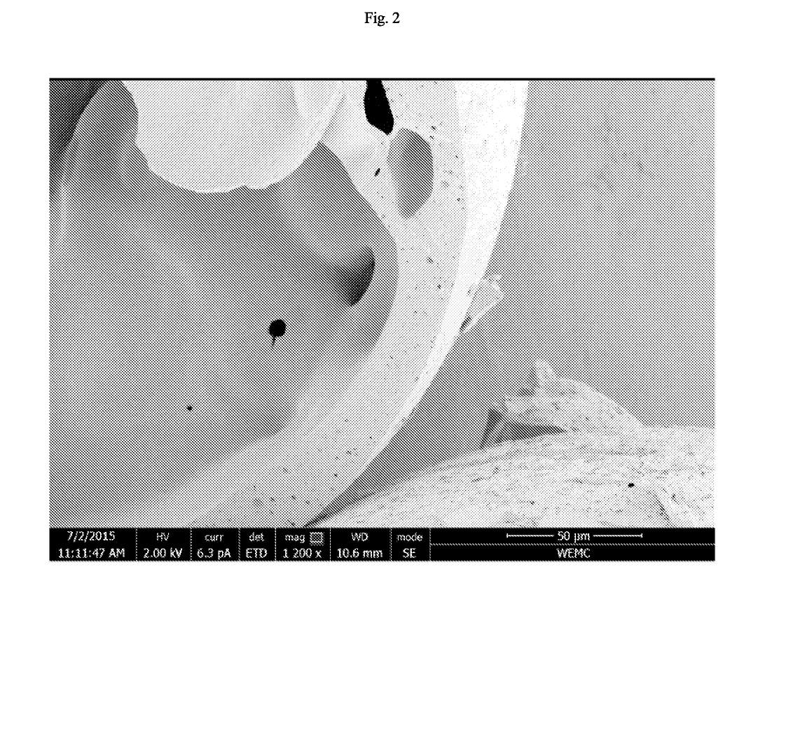

An example of such granule is shown in FIGS. 2 and 3, while FIG. 1 showing a non-popped granule prepared with the method of the present invention. As can be witnessed from said Figures, it was surprisingly found that, even though the granule was “popped”, the outer surface of said granule remain essentially closed, and displays a high density. In other words, at least 70%, at least 80% of the outer surface of the granule, preferably at least 90%, 95% of the outer surface of the granule is formed by the water-soluble dietary fiber and the functional substance, whereas at most 30%, 20%, 10%, 5% of the outer surface of the granule are openings in the surface giving access to the interior of the granule. In the practice of the present invention it is observed that most, if not virtually all intact granules display a 100% closed outer surface, and thus in one embodiment, the popped granule display a closed outer surface, with no openings.

Finally, there is provided for use of the granules and/or popped granules as described herein in the preparation of a food, a pet-food, feed, a cosmetic, a pharmaceutical, an edible composition, including a foam, an emulsion, a table and/or butter spread, cheese and imitated cheese, meat product, chocolate spread, filling, frosting, chocolate, confectionery, dairy product, frozen dessert, baked good, sauce, vegetables, vegetable meal mixes, fruit mixes, vegetable and fruit candies, soup, coffee whitener, and any other composition or product mentioned herein.

In addition, the granules and/or popped granules as described herein are useful in the preparation of products containing biologically active substances or organisms, such as yeast, bacteria, starter cultures, enzymes, therapeutic proteins (such as vaccines or antibodies), and other biological substances, including combinations thereof, as functional substance. It is particularly advantageous that these products can be stored during prolonged periods of time at ambient temperatures. Also, the biologically active substances retain most or all of their biological function and activity. For instance, a vaccine composition is envisaged which can easily be stored (dry and at ambient temperatures) and transported to a place of need.

Also provided is a composition comprising granules and or popped granules as described herein and/or as obtained or obtainable with the method of the present invention.

In summary, the present invention provides a simple and little energy consuming process to provide granules and popped granules. These granules and popped granules obtained with the method of the present invention have increased solubility and flowability and can release a better flavor, aroma, color and taste, while at the same time providing optimal nutrient retention and long shelf life. Due to the encapsulation, the granules or popped granules can deliver a higher quality authentic flavor and taste than the traditional commercial granules. The granules or popped granules exhibits a better stability of physical properties compared with existing granules. In particular, the granule or popped granule has a better (assured) flowability in time and a better solubility. The inventors found that flowability will not change in time and that there is a natural balance or no moisture migration between particles/granules)

Indeed, for specific products and applications, the granules, including popped granules, produced as described herein may, for example, have the following benefits:
Flavoring compounds (hydrophilic and lipophilic):
   Higher flavor compound load, for example 35% compared to current flavor encapsulation technologies;
   Lower process temperature enables better preservation of high volatile aroma compounds and biologically active substances;
   Flexibility in blending different flavor components with additional ingredients, such as vitamins, enzymes, phytonutrients, extracts, concentrates, vegetable and fruit purees, distillates, essences into one uniform granule;
   High flexibility in granule shape, i.e.: round, rectangular, flakes, compressed into chewy or hard candy-type
Extracts and distillates:
   Better retention of high volatile aroma compounds vs. spray drying;
   High flexibility in blending different extracts with flavors, vitamins, enzymes, phytonutrients, concentrates, essences into one uniform granulate;
Vitamins, enzymes, phytonutrients:
   Better retention of active compounds, because of lower process temperatures in contrast to, for example, spray drying;
Concentrates, essences:
   Clean label fruit-, vegetable-, herbal-, plant- and spice concentrates converted in granules, free flowing without free-flowing agents;
   Single- or multiple ingredient combinations preventing de-mixing in production
Vegetables, vegetable meal mixes, fruits, fruit mixes, herbs, herbs and spice mixes, soups, sauces, bouillons, condiments:
   Multiple fresh and or clean label ingredients blended in an emulsion and processed into one uniform granule;
   No de-mixing and free flowing without the use of free flowing agents;
   Without use of digestible saccharides, but with a low caloric water-soluble fiber;
Hot tea and coffee:
   Instant hot tea and flavoured teas or coffee and flavoured coffees without digestible saccharides; superior taste vs. instant hot tea or coffee made with e.g. digestible maltodextrin;
   Improved aroma profiles because of lower processing temperatures vs. spray dried techniques
Beverage compounds:
   Liquid beverage compounds made into quick dissolving granulate/powder with an all-in-one uniform structure;
   Without the use of digestible saccharides, reducing calories
Baby food:
   Long shelf life and affordable baby food by preserving fresh ingredient blend into granulation process according to the present invention.
   Improved color retention of ingredients because of low drying temperature and increased UV protection from the encapsulation properties of the digestion-resistant dextrin within the granulate structure.

In a particular preferred embodiment, the functional substance or substance of interest to be included in the granules as prepared by the method described herein comprises fresh fruit and/or vegetable. It was surprisingly found that with the invention the fresh fruit and/or vegetables are provided with an improved shell life when encapsulated in accordance with the method detailed herein. This is of significance in view of, for example, the large waste streams of fresh food and vegetables. With the current invention spoilage of such food and/or vegetables may be reduced while at the same time reducing the waste of such food components.

We have found that when using digestion-resistant dextrin or a digestion-resistant maltodextrine, with an average degree-of-polymerization of 4-30, for example 8-27 or 9-25, 4-10 or 12-25 and/or an average molecular weight of 3500-6500, for example 3500-4500 or 4000-6000 or combinations thereof, and in particular Nutriose and Fibersol-2, as water soluble dietary fibers, the above benefits are achieved. Using the method of the invention, for example by using Nutriose or Fibersol-2, we have been able to achieve a surprising level of retention of flavor, color, freshness and preservation of (biologically) active substances. Also, in complex mixtures of substances of interest, the granules of the invention contain an evenly distributed content of the different substances of interest, which is beneficial especially in food and pharmaceutical applications.

We also tested inulin (Frutafit CLR) and polydextrose (Sta-Lite L90) in the various experiments. However far less satisfactory results (particularly bad hygroscopicity and stickiness resulting in poor flowability of the granules and processing complexity) were obtained compared to the digestion-resistant dextrin and digestion-resistant maltodextrin (e.g. Nutriose and Fibersol-2 as disclosed herein) tested. Even in tests with increased dosages of inulin and polydextrose the flowability of the granules didn't perform as good as the digestion-resistant dextrin and digestion-resistant maltodextrin.

Having now generally described the invention, the same will be more readily understood through reference to the following examples which is provided by way of illustration and is not intended to be limiting of the present invention.

### EXAMPLES

### Example 1

### Fresh Garlic Granulate

Ingredients:

1. Nutriose FM06 (35% by weight) or Sta-Lite R90 (35% and 50% by weight)

2. Fresh garlic paste (40% by weight)

3. Water (25% by weight)

Mixing time: 2 minutes

Sheeting: 1.2 mm

Drying conditions: Oven; 30 degrees Celsius, 14 h

Moisture content after pre-drying: 10%

Dry granulating and sieving: granules size between 200-1000 micrometer.

Final product:
Nutriose FM06—solid crisp granulate, non-sticky and good flowability
Sta-Lite L90—difficult granulate processing because of hygroscopicity, less solid crisp granulate structure. Poor flowability even at a higher dosage of 50%

Nutriose FB06 was also tested with similar results as compared to Nutriose FM06.

### Example 2

### Popping the Nutriose-Based Granules of Example 1.

Expansion conditions (popping): hot plate for 6 sec or fluid bed at 150-180 degrees Celsius for 20-50 seconds.

Final product:
Nutriose FM06—Glassy, free flowing, uniform, dry round granules, with a fresh garlic flavor profile and an open inner structure. Excellent solubility, colour retention and flowability.
Sta-Lite L90—expansion step proved to be impossible

Nutriose FB06 was also tested with similar results as compared to Nutriose FM06.

### Example 3

### Low Salt Granulate Solution in Snack Application

Ingredients:

1. Nutriose FM06 (30% by weight); or Fibersol-2

2. Salt fine (50% by weight)

3. Water (20% by weight)

Mixing time: 2 minutes (hot water)

Sheeting: 1.2 mm

Drying conditions: 50 degrees Celsius for 4 hours or 30 seconds at 1000 Watt.

Moisture content after drying: 8%

Dry granulating and sieving: granules with a size of 200-1000 micrometer

Expansion conditions: hot plate for 7 sec

Final products: Glassy free flowing uniform dry round granules with a salty taste and open inner structure. Excellent solubility, both in expanded- and in unexpanded granulate formats. Nutriose FM06 delivered an overall better flowable expanded granule than the Fibersol-2 variant, although Fibersol-2 still being acceptable.

Experiment was repeated with inulin (Frutafit CLR). The somewhat softer granulate showed a slow solubility and unsatisfactory hygroscopicity. Expanding the granulate proved to be impossible.

Nutriose FB06 was also tested with similar results as compared to Nutriose FM06.

### Example 4

### Instant Soup Granulate

Ingredients:

1. Nutriose FM06 (digestion-resistant dextrin) or Fibersol-2 (digestion-resistant maltodextrin) (20% by weight)

2. Salt fine (30% by weight)

3. Maize starch (5% by weight)

4. Fresh Oregano chopped (10% by weight)

5. Fresh garlic paste (10% by weight)

6. Fresh onion paste (10% by weight)

7. Water (15% by weight)

Mixing and blending time: High sheer, 2 minutes (hot water)

Adding Maize starch after mixing and cooling solution

Sheeting: 1.2 mm

Drying conditions: 30 degrees Celsius for 4 hours.

Moisture content after pre-drying: 8%

Dry granulating and sieving: granules with a size of 200-1000 micrometer

Expansion conditions (popping): hot plate for 7 sec

Final product: Both Nutriose FM06 and Fibersol-2 delivered glassy, free flowing, dry, uniform round instant soup granules with a fresh taste and open inner structure. Excellent colour retention and hygroscopicity.

A similar test with Frutafit CLR didn't deliver satisfying results, caused by high level of stickiness during processing, with resulting granules showing hygroscopicity problems.

Nutriose FB06 was also tested with similar results as compared to Nutriose FM06.

### Example 5

### Tea Bag Fruit Flavoring

Ingredients:

1. Nutriose FM06 or FM10 (60% by weight), or maltodextrin (Maldex 190)

2. Topnote Orange (10% by weight)

3. Topnote Grapefruit (28% by weight)

4. Coloring system (2% by weight)

Mixing and blending time: High sheer, 2 minutes

Sheeting: 1.2 mm

Drying conditions: 30 degrees Celsius for 5 hours.

Dry granulating and sieving: granules with a size of 200-1000 micrometer

Final product:
Nutriose FM06: Glassy free flowing uniform granule with a firm structure. Excellent fresh flavor profile and hygroscopicity, no caking.
Nutriose FM10: glassy, free flowing, uniform granule, with a firm structure. Excellent fresh flavor profile and hygroscopicity, no caking
Maldex 190: significantly less firm granule structure, leading to more material dust. Also at a higher dosage of 70% by weight no improvement to achieve firmer structure.
Experiment was additionally repeated with inulin Frutafit CLR and polydextrose Sta-Lite L90, but the results were poor, mainly because of stickiness of the final structures resulting in caking of the, also less firm, granules.

Nutriose FB06 was also tested with similar results as compared to Nutriose FM06.

### Example 6: Instant Coffee Milk Granulate

Ingredients:

1. Nutriose FM06, Inuline Frutafit CLR, or a mixture (1:1) of both (10% by weight).

2. Liquid coffee creamer (90% by weight)

Mixing and blending time: 2 minutes

Sheeting: 0, 8 mm

Drying conditions: 30 degrees Celsius for 12 hours.

Dry granulating and sieving: granules with a size of 200-1000 micrometer

Final products:
Nutriose FM06: glassy uniform granule with excellent hygroscopy and free flowing with a firm structure that retains natural milk aroma profile.
Frutafit CLR: stickiness during processing. Resulting in caking granules with slow solubility
Nutriose FM06 mixed with Frutafit CLR (1:1): no problems in granule processing or solubility, but some caking was noticed.

Nutriose FB06 was also tested with similar results as compared to Nutriose FM06.

### Example 7

### Heat Stable Lemon Granulate for Battered Coated Fried Potato

Ingredients:

1. Nutriose FM06 (60% by weight)

2. Topnote Lemon (30% by weight)

3. Palm fat: (10% by weight)

Mixing and blending time: 2 minutes

Sheeting: 0.8 mm

Drying conditions: 30 degrees Celsius, 5 hours.

Dry granulating and sieving: granules with a size of about 700-2000 micrometer

Encapsulation: Glatt Spray granulation, hardened palmfat (Cessa powder 60 Karlshamns)

Final product: Glassy free flowing uniform, Non-water soluble granule with fresh lemon taste and firm structure.

Nutriose FB06 was also tested with similar results as compared to Nutriose FM06.

### Example 8

### Dried Bakers Yeast

Ingredients:

1. Nutriose FM06 (50% by weight)

2. Wet bakers yeast DSM (contains ±90% water): (50% by weight)

Mixing and blending time: 2 minutes

Sheeting: 0.8 mm

Drying conditions: 30 degrees Celsius, 5 hours.

Dry granulating and sieving: granules with a size of about 700-2000 micrometer

After storage of the final product for 6 weeks, the product was rehydrated and used to bake bread. Yeast performed well.

Nutriose FB06 was also tested with similar results as compared to Nutriose FM06.

### Example 9

### Vitamin for Bread Improver

Ingredients:

1. Nutriose FM06 (60% by weight)

2. Ascorbic Acid Vitamin C

Mixing and blending time: 2 minutes

Sheeting: 0.8 mm

Drying conditions: 30 degrees Celsius, 5 hours.

Dry granulating and sieving: granules with a size of about 700-2000 micrometer.

After storage of the final product for 6 weeks, the bread improver vitamin C was rehydrated. This dried vitamin has an effect on dough and bread properties.

Nutriose FB06 was also tested with similar results as compared to Nutriose FM06.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

1. A method for preparing granules comprising a substance of interest and a water-soluble dietary fiber being a digestion-resistant dextrin or digestion-resistant maltodextrin, the method comprising the steps of:
a) preparing an aqueous mixture comprising said substance of interest and said water-soluble dietary fiber;
b) drying the mixture obtained in step a) at a temperature of between 10-80 degrees Celsius until the mixture adopts a glassy structure; and
c) forming granules from the glassy structure obtained in step b).

2. The method of claim 1 wherein the method further comprises the step of
d) popping at least part of the granules obtained in step c) to obtain popped granules, preferably wherein popping is performed by use of a fluid bed dryer, a plate dryer, a microwave, or by applying radiation and/or wherein popping is performed under reduced pressure.

3. The method of any one of the preceding claims wherein the water-soluble dietary fiber is Nutriose or Fibersol-2.

4. The method of any one of the preceding claim wherein the water-soluble dietary fiber is a digestion-resistant dextrin with an average degree-of-polymerization of 4-30, 8-27 or 9-25, preferably 4-10 or 12-25 and/or an average molecular weight of 3500-6500, 3500-4500 or 4000-6000 dalton.

5. The method of any one of the previous claims wherein the mixture prepared in step a) is a nonflowable mixture and/or wherein step a) comprises removing water from the mixture prepared in step a) to provide a nonflowable mixture.

6. The method of any one of the previous claims wherein the mixture prepared in step a) is formed into a shape, preferably a sheet, preferably by casting or injection molding.

7. The method of claim 6 wherein the sheet is provided with a sheet thickness of, with increasing preference, between 0.01-20 cm, between 0.05-10 cm, between 0.1-4 cm, or between 0.1-2 cm.

8. The method of any one of the preceding claims wherein the substance of interest is a food substance, a feed substance, a pharmaceutical substance, a cosmetic substance, or a biologically active substance or organism, wherein the organism preferably is yeast, bacteria, starter cultures, enzyme, therapeutic protein preferably being a vaccine or antibody, including combinations thereof.

9. The method of any one of the preceding claims wherein the aqueous mixture in step a) is prepared by mixing said substance of interest with said water-soluble dietary fiber in the presence of water, preferably wherein the mixing is at a temperature of between 10-80 degrees Celsius.

10. The method of any one of the preceding claims wherein more than one type of water-soluble dietary fiber is used in preparing the aqueous mixture in step a).

11. The method of any one of the preceding claims wherein in step a) said at least one substance of interest and at least one water-soluble dietary fiber are mixed in a ratio of between 1:20-20:1.

12. The method of any one of the preceding claims wherein in step b) drying is by convection drying, conduction drying, radiation drying, microwave drying or combinations thereof.

13. The method of any one of the preceding claims wherein in step b) drying is at a temperature of, with increasing preference, at most 80 degrees Celsius, at most 70 degrees Celsius, at most 60 degrees Celsius, at most 50 degrees Celsius, at most 40 degrees Celsius, at most 30 degrees Celsius, or at most 20 degrees Celsius.

14. The method of any one of the preceding claims wherein in step b) drying is under conditions with a relative humidity of, with increasing preference, at most 50%, at most 40%, at most 30%, or at most 20%.

15. The method of any one of the preceding claims wherein in step b) the glassy structure has a water content of, with increasing preference, between 0.01-15% (w/w), between 0.05-10% (w/w), between 0.1-8% (w/w) or between 0.1-5% (w/w).

16. The method of any one of the preceding claims wherein in step c) the forming of granules is by breaking or milling the glassy structure obtained in step b).

17. The method of any of the preceding claims wherein the popped granules obtained in step d) have a water content of, with increasing preference, between 0.001-12% (w/w), between 0.005-11% (w/w), between, or 0.01-10% (w/w).

18. A granule or popped granule obtainable with the method of any one of claims 1-17.

19. A granule or popped granule, comprising, by weight,
1-99%, preferably 40-99%, even more preferable 75-99% or 40-75% substance of interest;
1-99%, preferably 1-60%, even more preferably 1-25% or 25-60% water-soluble dietary fiber being a digestion-resistant dextrin or digestion-resistant maltodextrin; and
0-15% water, and wherein substance of interest, water-soluble dietary fiber and water add up to no more than 100%.

20. A popped granule according to claim 19, wherein the popped granule is a granule characterized by an outer surface that is substantially closed and an interior with a cellular structure or meshwork structure with multiple pores or cavities dispersed within the meshwork, preferably a honeycomb structure, preferably wherein the granule has a size of between 50 micrometer and 5000 micrometer, more preferably between 100 micrometer and 2000 micrometer, preferably wherein the pores or cavities in the interior are present in a quantity of 15 to 95%, for example 15 to 75%, by volume.

21. A method according to claims 1-17 or the granules as defined in any of claims 18-20, characterized in that the substance of interest is a biologically active compound or organism preferably being a protein (preferably a vaccine or an antibody, vitamin, yeast, bacteria or combinations thereof.

22. Use of granules obtainable by the method of any one of 1-17 or the granules as defined in any of claims 18-20 in the preparation of a food, a pet-food, feed, a cosmetic, a pharmaceutical, an edible composition, including a foam, an emulsion, a table and/or butter spread, cheese and imitated cheese, meat product, chocolate spread, filling, frosting, chocolate, confectionery, dairy product, frozen dessert, baked good, sauce, soup, and coffee whitener.

23. A composition comprising granules and or popped granules according to any one of claims 18-20, and/or obtainable or obtained with any one of the methods of claim 1-17 or 21.
